(19) United States
(12) Patent Application Publication
(10) Pub. No.: US 2026/0151406 A1
(43) Pub. Date: Jun. 4, 2026

(54) COMPOSITION FOR PREVENTING OR TREATING PERIODONTITIS COMPRISING CANNABIDIOL AND TAURINE

(71) Applicant: MBP CO., LTD., Jeonju-si, Jeollabuk-do (KR)

(72) Inventors: Yun-jo SOH, Jeonju-si (KR); Hyun-joo SHIM, Yongin-si (KR); Chang-jin IM, Jeonju-si (KR)

(73) Assignee: MBP CO., LTD., Jeonju-si, Jeollabuk-do (KR)

(21) Appl. No.: 19/123,582

(22) PCT Filed: Aug. 4, 2023
(86) PCT No.: PCT/KR2023/011476

§ 371 Date: Apr. 23, 2025

(30) Foreign Application Priority Data
Oct. 24, 2022 (KR) 10-2022-0137558

(51) Int. Cl. A61K 31/00; A61K 9/00; A61K 31/185; A61P 1/02

(52) U.S. Cl. CPC A61K 31/658; A61K 9/0056; A61K 31/185; A61P 1/02

(57) ABSTRACT

The present invention relates to a composition for preventing or treating periodontitis containing cannabidiol and taurine. The composition according to the present invention can effectively prevent or treat periodontitis by suppressing inflammatory factors and directly suppressing the loss of alveolar bone. In addition, it is safe for the human body and has almost no side effects, so it can be used in various ways as a material for medicines, quasi-drugs, or health functional foods.

### TECHNICAL FIELD

This application claims priority under Korean Patent Application No. 10-2022-0137558, which was filed on Oct. 24, 2022, and the entire disclosure of which is incorporated herein by reference.

The present invention relates to a composition for preventing or treating periodontitis comprising cannabidiol and taurine.

### BACKGROUND ART

Periodontitis is a chronic inflammatory disease of the periodontium characterized by destruction of connective tissue and loss of alveolar bone. The main etiology of periodontal tissue destruction is dental biofilm containing periodontogenic bacteria, which induces a host immune response. For example, the gram-negative anaerobic bacterium, Porphyromonas gingivalis, one of the major pathogens of periodontitis, releases lipopolysaccharide (LPS) from its outer membrane vesicles. LPS stimulates macrophages to secrete proinflammatory mediators, including tumor necrosis factor-α (TNF-α), interleukin-1β (IL-1β), cyclooxygenase (COX), inducible nitric oxide synthase (iNOS), and matrix metalloproteinases (MMPs). Most of these molecules act as potential mediators of alveolar bone resorption in periodontitis.

In periodontal research, ligation-induced periodontitis has been developed using animals. For example, rodent models have been widely used to investigate periodontal disease mechanisms. Ligation induces localized accumulation of bacteria in the subgingival region and enhances bacteria-mediated inflammation and bone loss.

Cannabidiol (CBD) is a substance isolated and extracted from cannabis. It is a major physiologically active substance of cannabis along with THC, and has been reported to be effective for various diseases. In particular, CBD-related receptors have been reported to play an important role in bone formation, so CBD is known to indirectly act on bone loss inhibition and bone formation. In addition, CBD plays an important role in suppressing inflammation of rheumatoid arthritis synovial fibroblasts, and suppresses important cytokines (IL-1b, TNF-a, iNOS, Cox-2, etc.) related to inflammation in periodontitis. However, there are no reports that it suppresses inflammation caused by direct dental disease and periodontal bone loss at the same time.

Meanwhile, taurine is known to have many physiological functions. For example, taurine is currently known to be involved in osmoregulation, membrane stabilization, calcium mobilization, neurotransmission, reproduction, and detoxification. In particular, taurine has been reported to provide anti-inflammatory effects and protect cells from the cytotoxic effects of inflammation. However, there are only few reports on the effectiveness of taruine in treating periodontitis.

### DISCLOSURE

### Technical Problem

The present invention provides a pharmaceutical composition for preventing or treating periodontitis, comprising cannabidiol and taurine, or a pharmaceutically acceptable salt thereof, as active ingredients.

In addition, the present invention provides a quasi-drug composition for preventing or ameliorating periodontitis, comprising cannabidiol and taurine, or a pharmaceutically acceptable salt thereof, as active ingredients.

In addition, the present invention provides a food composition for preventing or ameliorating periodontitis, comprising cannabidiol and taurine, or a pharmaceutically acceptable salt thereof, as active ingredients.

However, the technical problems to be solved by the present invention are not limited to the problems mentioned above, and other problems not mentioned will be clearly understood by those skilled in the art from the description below.

### Technical Solution

The present invention provides a pharmaceutical composition for preventing or treating periodontitis, comprising cannabidiol and taurine, or a pharmaceutically acceptable salt thereof, as active ingredients.

The cannabidiol can be represented by the following chemical formula 1:

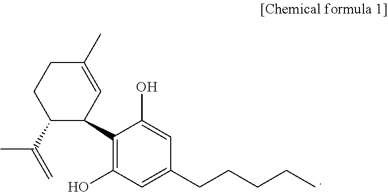 [SMILES: C=C(C)[C@@H]1CCC(C)=C[C@H]1c1c(O)cc(CCCCC)cc1O]

The taurine can be represented by the following chemical formula 2:

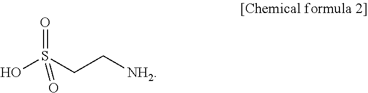

The present effective ingredients can inhibit TNF-α and IL-1β.

In addition, the present invention provides a quasi-drug composition for preventing or ameliorating periodontitis, comprising cannabidiol and taurine, or a pharmaceutically acceptable salt thereof, as active ingredients.

In addition, the present invention provides a food composition for preventing or ameliorating periodontitis, comprising cannabidiol and taurine, or a pharmaceutically acceptable salt thereof, as active ingredients.

In addition, the present invention provides a method for ameliorating or treating periodontitis, comprising a step of administering or ingesting a composition comprising cannabidiol and taurine, or a pharmaceutically acceptable salt thereof, as active ingredients to a subject in need thereof.

In addition, the present invention provides a use of a composition comprising cannabidiol and taurine, or a pharmaceutically acceptable salt thereof, as active ingredients for preventing, ameliorating or treating periodontitis.

### Advantageous Effects

The composition according to the present invention can effectively prevent, ameliorate or treat periodontitis by suppressing inflammatory factors and directly suppressing the loss of alveolar bone. In addition, the present composition is safe for the human body and has almost no side effects, so it can be used in a variety of ways as a material for medicines, quasi-drugs, or health functional foods.

### DESCRIPTION OF DRAWINGS

FIG. 1 shows the results of evaluating the intracellular toxicity of cannabidiol and taurine.

FIG. 2 shows the analysis of the inhibitory efficacy of cannabidiol and taurine on inflammatory factors, iNOS and COX-2, when treated alone or in combination.

FIG. 3 shows the analysis of the inhibitory efficacy of cannabidiol and taurine on the formation of TRAP-positive cells in macrophages when treated alone or in combination (***, P<0.001).

FIG. 4 shows the analysis of efficacy of inhibiting bone loss of cannabidiol and taurine when treated alone or in combination (***, P<0.001).

FIG. 5 shows the analysis of efficacy of inhibiting pocket depth due to periodontitis by the combined treatment of cannabidiol and taurine (***, P<0.001).

FIG. 6 shows the analysis of efficacy of inhibiting alveolar bone loss due to periodontitis by the combined treatment of cannabidiol and taurine (*, P<0.05; **, P<0.01; ***, P<0.001).

### MODE OF THE INVENTION

The present inventors have found that cannabidiol and the present invention was completed by experimentally confirming that the combined treatment with taurine was significantly effective in preventing or treating periodontitis.

The development of periodontitis treatments developed to date has focused on treating periodontitis through inflammation suppression, but because periodontitis is very difficult or impossible to recover from bone loss caused by inflammation, prevention through inflammation suppression is important, but treatment should be developed in a way that minimizes the problem of alveolar bone loss.

The present invention can effectively treat periodontitis by suppressing inflammatory factors and directly suppressing alveolar bone loss, and thus can be usefully utilized as a material for pharmaceuticals, quasi-drugs, or health functional foods.

Hereinafter, the present invention will be described in detail.

The present invention provides a composition for preventing (or ameliorating) or treating periodontitis, comprising cannabidiol and taurine, or a pharmaceutically acceptable salt thereof, as active ingredients.

The effective ingredients have the effect of suppressing inflammatory factors such as TNF-α and IL-1β.

The composition may be a pharmaceutical composition, a quasi-drug composition, a food composition or a health functional food composition.

The composition may include cannabidiol:taurine in a concentration ratio of 1:5 to 50.

Cannabidiol (CBD) is a plant cannabinoid discovered in 1940, and is one of 113 cannabinoids identified in the plant Hemp. As of 2018, preliminary clinical studies of cannabidiol have included studies of anxiety, cognition, and movement disorders. In the United States, a cannabidiol drug called Epidiolex has been approved by the U.S. Food and Drug Administration to treat epilepsy.

The cannabidiol can be represented by the following chemical formula 1:

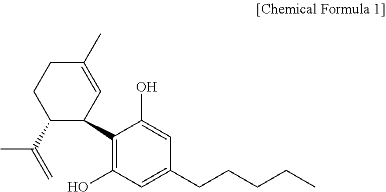 [SMILES: C=C(C)[C@@H]1CCC(C)=C[C@H]1c1c(O)cc(CCCCC)cc1O]

Taurine is an amine containing sulfur that exists in the cells and tissues of mammals including humans, and is mainly found in shellfish such as oysters, scallops, squid, octopus, and the dark red flesh of fish. Taurine is also known to be very safe, and one of the features that differentiates it from other amino acids is that even if consumed in excess, there have been no reports of decreased absorption, growth inhibition, or other side effects.

The taurine can be represented by the following chemical formula 2:

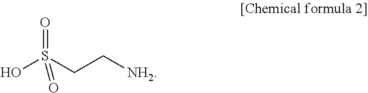

The method for preparing or obtaining cannabidiol and taurine of the present invention is not particularly limited, and can be separated from natural products, chemically synthesized using a known method, or commercially available products can be used.

In the present invention, cannabidiol and taurine may include hydrates, derivatives, etc. within the range having the same efficacy, and may include solvates or stereoisomers thereof.

In the present invention, the term “prevention” means any action that delays the onset of periodontitis by administering the composition of the present invention, and “treatment” and “ameliorate” mean any action that improves or beneficially changes the symptoms of periodontitis by administering the composition of the present invention.

In the present invention, the term “pharmaceutically acceptable salt” or “salt thereof” may be an acid addition salt formed by a free acid. The acid addition salt may be prepared by conventional methods, for example, by dissolving the compound in an excess of an aqueous acid solution and precipitating the salt using a water-miscible organic solvent, for example, methanol, ethanol, acetone or acetonitrile. Also, a pharmaceutically acceptable salt may be prepared by mixing an equimolar amount of the compound and an acid or alcohol (for example, glycol) in water. The free acid may be an inorganic acid or an organic acid. Non-limiting examples of the inorganic acid include hydrochloric acid, phosphoric acid, sulfuric acid, nitric acid, tartaric acid, etc., and these may be used alone or in combination of two or more. The “pharmaceutically acceptable salt” or “salt thereof” may be an acid addition salt formed by a free acid. The acid addition salt may be prepared by a conventional method, for example, dissolving the compound in an excess amount of an aqueous acid solution and precipitating the salt using a water-miscible organic solvent, for example, methanol, ethanol, acetone or acetonitrile. In addition, an equimolar amount of the compound and an acid or alcohol (for example, glycol monomethyl ether) in water can be heated and the mixture can then be evaporated to dryness, or the precipitated salt can be filtered off with suction.

The salt of cannabidiol and taurine may include all salts of acidic or basic groups that may be present in the compounds of cannabidiol and taurine, unless otherwise indicated. For example, the salt of cannabidiol and taurine may include sodium, calcium or potassium salt of hydroxyl group, and other pharmaceutically acceptable salt of amino group may include hydrobromide, sulfuric acid, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate) salt, and can be produced using the methods known in the industry for producing salts.

### Pharmaceutical Composition Comprising Cannabidiol and Taurine, or Pharmaceutically Acceptable Salts Thereof, as Active Ingredients

The composition of the present invention can be prepared as a pharmaceutical composition.

If the composition of the present invention is prepared as a pharmaceutical composition, the pharmaceutical composition of the present invention may include a pharmaceutically acceptable carrier.

According to a preferred embodiment of the present invention, the composition of the present invention may be a pharmaceutical composition comprising (a) a pharmaceutically effective amount of the cannabidiol and taurine of the present invention; and (b) a pharmaceutically acceptable carrier. The term “pharmaceutically effective amount” as used herein means an amount sufficient to achieve the efficacy or activity of the cannabidiol and taurine as described above.

Pharmaceutically acceptable carriers are commonly used, such as lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl Cellulose, methylhydroxybenzoate, propylhydroxybenzoate, talc, magnesium stearate, and mineral oil, but are not limited thereto. The pharmaceutical composition of the present invention may further contain, in addition to the components, a lubricant, a humectant, a sweetening agent, a flavoring agent, an emulsifier, a suspending agent, a preservative, and the like. Suitable pharmaceutically acceptable carriers and formulations are described in detail in Remington's Pharmaceutical Sciences (19th ed., 1995).

The pharmaceutical composition of the present invention can be administered orally or parenterally.

The dosage of the pharmaceutical composition of the present invention can be prescribed in various ways depending on factors such as the formulation method, administration method, patient's age, weight, sex, pathological condition, food, administration time, administration route, excretion rate, and reaction sensitivity. The general dosage of the pharmaceutical composition of the present invention is within the range of 0.001-100 mg/kg for adults. Administration may be once a day or divided into several times. However, the scope of the present invention is not limited by the dosage.

The pharmaceutical composition of the present invention can be manufactured in a unit dosage form or can be manufactured by placing it in a multi-dose container by formulating it using a pharmaceutically acceptable carrier and/or excipient according to a method that can be easily performed by a person having ordinary skill in the art to which the present invention pertains, and by putting it in a multi-dose container. At this time, the formulation may be in the form of a solution, suspension, syrup or emulsion in an oil or aqueous medium, or may be in the form of an extract, powder, granule, tablet or capsule, and may additionally contain a dispersant or stabilizer.

### Quasi-Drug Composition Comprising Cannabidiol and Taurine, or a Pharmaceutically Acceptable Salt Thereof, as Active Ingredients

The composition of the present invention can be provided as a quasi-drug composition.

The above-mentioned effective ingredients of the present invention may be added as they are, or other ingredients commonly used in oral quasi-drug composition may be included, such as abrasives, wetting agents, binders, foaming agents, sweeteners, preservatives, medicinal ingredients, flavoring agents, coloring agents, solvents, whitening agents, solubilizers, or pH adjusters. The mixing amount of the effective ingredients may be appropriately determined depending on the purpose of use (prevention, health, or therapeutic treatment).

The quasi-drug composition may be manufactured in any dosage form commonly manufactured in the art, and may have dosage forms such as, but not limited to, toothpaste, mouthwash, mouthwash, gum, candy, mouth spray, oral ointment, oral varnish, mouthwash, and gum massage cream.

As an example, when the quasi-drug composition of the present invention is in the form of a toothpaste, it may include a wetting agent, an abrasive, a binder, a foaming agent, a flavoring agent, a sweetener, a coloring agent, a preservative, a pharmaceutical ingredient, a solvent, a pH regulator, etc.

### Food Composition Comprising Cannabidiol and Taurine, or Pharmaceutically Acceptable Salts Thereof, as Active Ingredients

The composition of the present invention can be provided as a food composition or a health functional food composition. When the composition of the present invention is manufactured as a food composition, it includes not only the cannabidiol and taurine as active ingredients, but also components commonly added during food manufacturing, such as proteins, carbohydrates, fats, nutrients, seasonings, and flavoring agents. Examples of the carbohydrates described above include monosaccharides, such as glucose, fructose, etc.; disaccharides, such as maltose, sucrose, oligosaccharides, etc.; and polysaccharides, such as dextrin, cyclodextrin, etc., and sugar alcohols, such as xylitol, sorbitol, and erythritol. As flavoring agents, natural flavoring agents [thaumatin, stevia extracts (e.g., rebaudioside A, glycyrrhizin, etc.)] and synthetic flavoring agents (saccharin, aspartame, etc.) can be used. For example, when the food composition of the present invention is manufactured into a drink, citric acid, liquid fructose, sugar, glucose, acetic acid, malic acid, juice, Eucommia ulmoides extract, jujube extract, licorice extract, etc. may be additionally included in addition to the active ingredients of the present invention.

The formulation of the food composition or health functional food composition can be in the form of powder, granules, pills, tablets, capsules, or in the form of general food or beverage.

There is no particular limitation on the type of the food, and examples of foods to which the active ingredients can be added include meat, sausage, bread, chocolate, candy, snacks, confectionery, pizza, ramen, other noodles, gum, dairy products including ice cream, various soups, beverages, tea, drinks, alcoholic beverages, and vitamin complexes, and all foods in the conventional sense can be included.

In general, when manufacturing food or beverage, the effective ingredient may be added in an amount of 15 parts by weight or less, preferably 10 parts by weight or less, per 100 parts by weight of the active ingredients. However, in the case of long-term intake for the purpose of health and hygiene or health control, the amount may be less than the above range, and since the present invention does not have any safety issues since it utilizes safe ingredients, it may be used in an amount greater than the above range.

Hereinafter, the present invention will be described in more detail through examples. These examples are only intended to explain the present invention more specifically, and it will be apparent to those skilled in the art that the scope of the present invention is not limited by these examples.

### EXAMPLES

Cannabidiol (CBD) was synthesized from olivetol and camphor according to a previously known synthetic method (V. Vaillancourt and K F Albizati, 1992). Taurine was purchased from (Sigma-Aldrich, Korea) and used.

### Example 1. Evaluation of Cytotoxicity

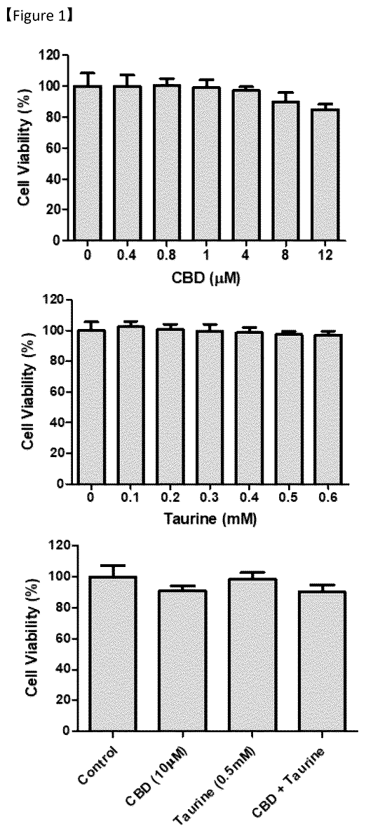

For CBD and taurine, intracellular toxicity evaluation was performed on osteoclasts (Raw264.7 cells), which are inflammatory cells, and the results are shown in FIG. 1. In the case of CBD, it was found that there was no intracellular toxicity up to a concentration of 12 uM, and in the case of taurine, it was confirmed that there was no intracellular toxicity up to a concentration of 0.6 mM. The appropriate concentrations of CBD (10 uM) and Taurine (0.5 mM) were determined, and intracellular toxicity evaluation according to subsequent combined treatment was performed. As a result, it was confirmed that there was no intracellular toxicity in the combination of CBD and taurine.

### Example 2. Analysis of Inhibitory Efficacy on Inflammation-Related Factors

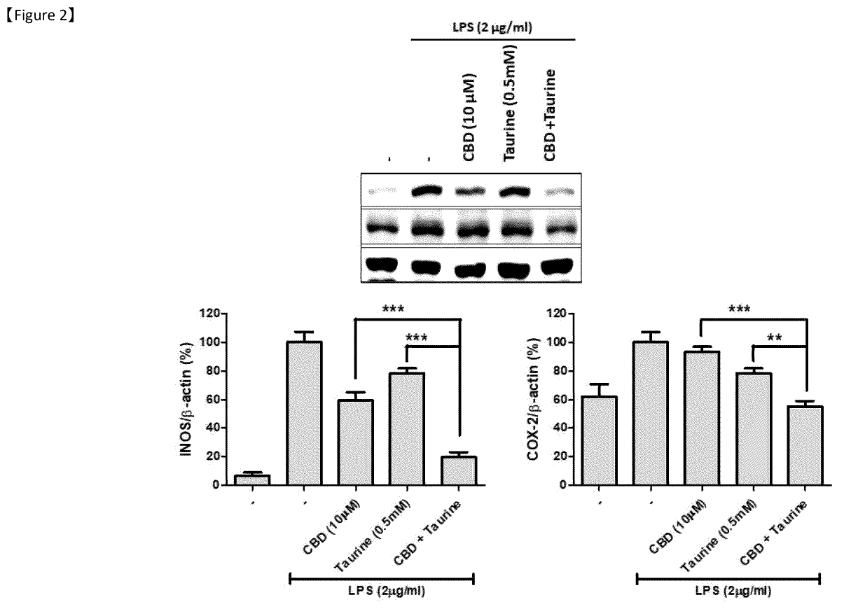

The inhibitory effects of CBD and taurine on COX-2 and INOS, which are inflammation-related factors. We treated macrophage RAW264.7 cells with LPS, an inflammation-inducing substance, and analyzed the anti-inflammatory effects of CBD and taurine alone and the anti-inflammatory effects according to combined treatment. As a result of the analysis, it was confirmed that the inhibitory efficacy of iNOS and the inhibitory efficacy of COX-2 were significantly increased when CBD and taurine were used in combination (FIG. 2).

### Example 3. Analysis of Inhibitory Efficacy on Bone Loss

To confirm the inhibitory effect of CBD and taurine on bone loss caused by periodontitis, the degree of TRAP formation and bone loss by RANKL (receptor activator of nuclear factor-KB ligand) was analyzed through pit formation area analysis.

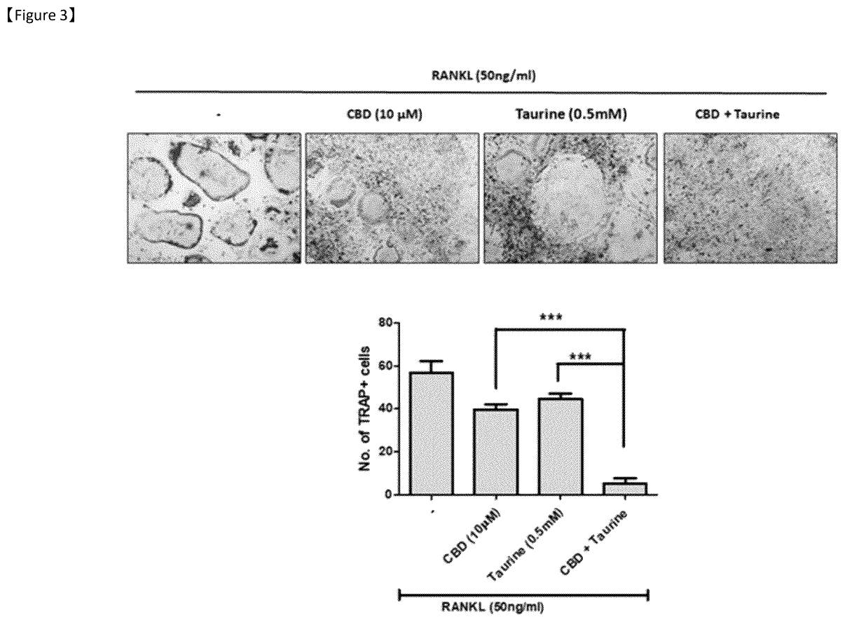

As a result of analyzing the degree of inhibition of TRAP-positive cell formation by RANKL during osteoclast differentiation, it was confirmed that the inhibition efficacy of TRAP-positive cell formation was very low when CBD or taurine was used alone, but the inhibition efficacy of TRAP-positive cell formation was very high when CBD and taurine were used in combination (FIG. 3).

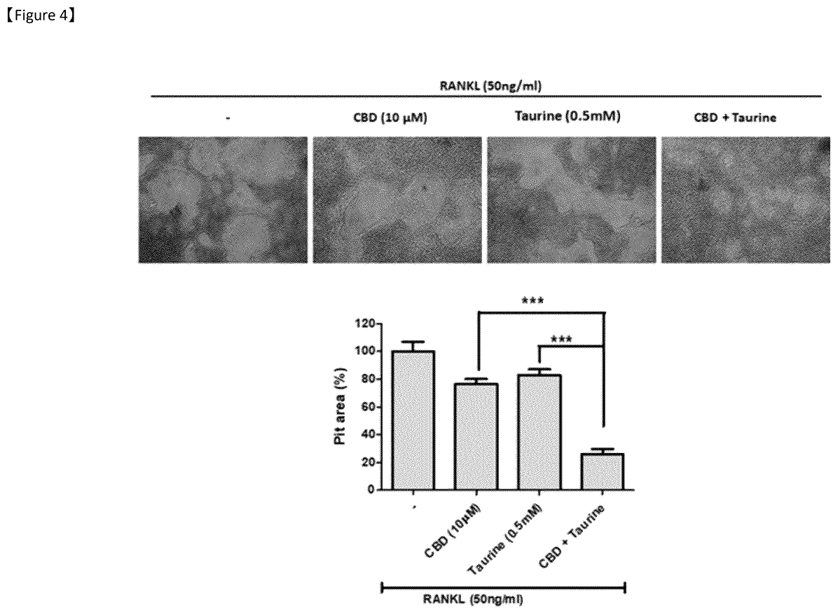

To further confirm the bone loss inhibition efficacy of CBD and taurine, the degree of inhibition of bone loss by RANKL in macrophages was analyzed. When CBD and taurine were used alone, the bone loss inhibition efficacy was almost none or very low, but when CBD and taurine were used together, the bone loss inhibition efficacy was confirmed to be very high (FIG. 4).

Therefore, it was found that when CBD and taurine were used together, the effect of suppressing inflammatory factors and inflammatory periodontitis was very excellent, and the effect of suppressing alveolar bone loss caused by inflammation was also very excellent.

### Example 4. Analysis of the Efficacy of Combined Administration of CBD and Taurine in Suppressing Periodontitis Using an Animal Model

### [Experimental Method]

### Preparation of Rats for Test

Rats were purchased from Nara Biotech Pyeongtaek Plant (Gyeonggi-do, Korea), and 6-week-old male Sprague-Dawley rats (M/6W, 180-200 g) were used for the test. Animal experiments were conducted in accordance with the regulations of the Institutional Animal Care and Use Committee (IACUC guidelines) of JEONBUK National University.

### Stabilization Period

During the stabilization period, the rats were allowed to adapt to the experimental environment for one week in an animal enclosure maintained at 20-24° C., humidity 50-60%, and day/night cycles of 12 hours each, with free access to food and water.

### Cause of Periodontitis

1) Preparation of Periodontitis-Causing Bacteria (P. gingivalis)

The periodontitis-causing bacteria (P. gingivalis) was obtained from the Korean Oral Microbial Resource Bank (PG 2797). The periodontitis-causing bacteria were prepared by inoculating them into the culture medium in an anaerobic state three weeks before the start of the experiment and incubating them for three weeks. The periodontitis-causing bacteria cultured for 3 weeks were visually confirmed using the turbidity of the culture solution and the growth rate of the bacteria inoculated onto the culture dish, and were then used to induce periodontitis for 7 days.

### 2) Cause of Periodontitis

Periodontitis was induced by ligature using a band treated with periodontitis-causing bacteria (TP Orthodontics, Inc., Seoul, Korea). The band suture was kept in a suspension containing periodontitis-causing bacteria so that the periodontitis-causing bacteria were well mixed in the band suture before treatment. The ligature band was removed to an appropriate size and ligated to the anterior region of the premolar of the mandibular quadrant to induce periodontitis. Three days later, the loss of the ligature band was confirmed, and the ligature band was reinserted into the lost region to induce periodontitis for seven days.

### Dosage

### 1) Dosage Method

After 7 days of inducing periodontitis, the test substance was orally administered daily for 2 weeks after measuring whether periodontitis had been induced using Pocket Depth. In order to prevent interference during administration (the test substance getting on the mouth when administered intragastrically and the test substance remaining in the syringe), the substance on the syringe was removed with tissue paper before administration, and the syringe was washed 3-5 times in water before administration of the test substance to remove any residual substance.

The total number of mice used in the experiment was 50, and 50 mice in 5 treatment groups (10 mice per each treatment group) were used in the experiment. The control group was a healthy group that has not been induced with periodontitis, and the vehicle group was a control group that was administered only water without any medication after periodontitis was induced. In addition, Insadol (4.5 mg/kg Dongkook Pharmaceutical, Korea) was purchased commercially and used as a control group and administered by dissolving in water. To examine the efficacy of periodontitis treatment according to the concentration of CBD, the low dose CBD+Taurine (L-CBD+Taurine) and high dose CBD+Taurine (H-CBD+Taurine) treatment groups were administered at a fixed concentration by dissolving Taurine (100 mg/kg Sigma-Aldrich, Seoul, Korea) in water. Since CBD does not dissolve in water, it was dissolved in Corn oil (Sigma-Aldrich) and administered at the concentrations of L-CBD (2 mg/kg) and H-CBD (20 mg/kg), respectively.

### Pocket Depth Measurement

Pocket depth was measured using a pocket depth probe. The effectiveness of inhibiting periodontitis was measured using the length of the pocket depth when the test substance was administered after the induction of periodontitis. The efficacy of inhibiting periodontitis was examined by measuring the pocket depth on three occasions: on the 7th, 14th, and 21st day after the induction of periodontitis.

### Micro-CT Analysis for Measuring Alveolar Bone Loss

Aleveolar bone loss was measured using a high-resolution in vivo X-ray microtomograph (HR Micro-CT) (Bruker MicroCT, Kontich, MA, USA) in the JEONBUK National University Joint Laboratory. After inducing total periodontitis, the test substance was administered until day 20, and on day 21, sacrificing was performed. The right maxillary bone was sampled and micro-CT was measured. Bone mineral density (BMD) was measured for each treatment group using two-dimensional measurement software (Bruker dataviewer, USA).

### [Experimental Results]

### Analysis of the Efficacy of Combined Treatment of CBD and Taurine to Suppress Pocket Depth Caused by Periodontitis

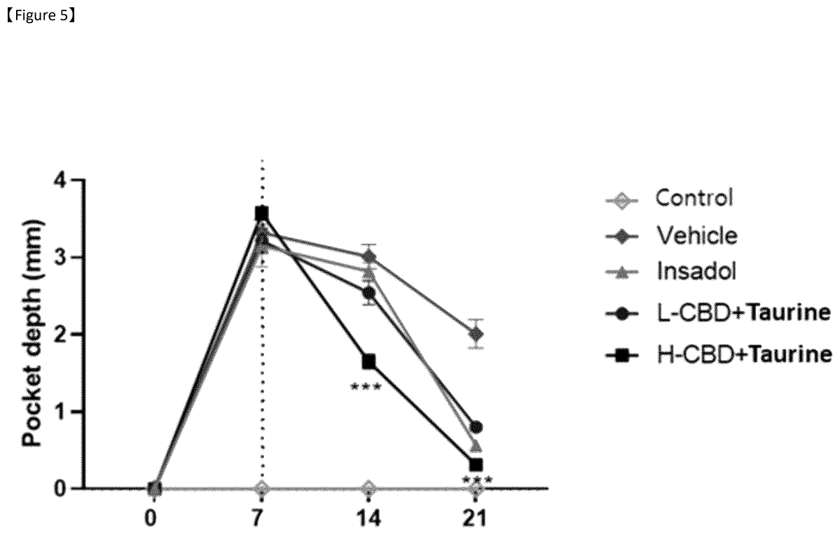

To examine the inhibition efficacy on periodontitis according to the concentration when CBD and Taurine were administered together, pocket depth was measured and the results are shown in FIG. 5. As a result of the measurement, the H-CBD+Taurine group showed the highest inhibition of pocket depth for 7 days from 7 days after periodontitis induction to 14 days, and the H-CBD+Taurine group also showed the highest inhibition of pocket depth on the 21st day (***, P<0.001). CBD showed the highest periodontitis inhibition efficacy when treated at a high concentration, and it was confirmed to show a higher periodontitis inhibition efficacy than Insadol in particular.

### Analysis of the Efficacy of Combined Treatment of CBD and Taurine to Suppress Alveolar Bone Loss Caused by Periodontitis

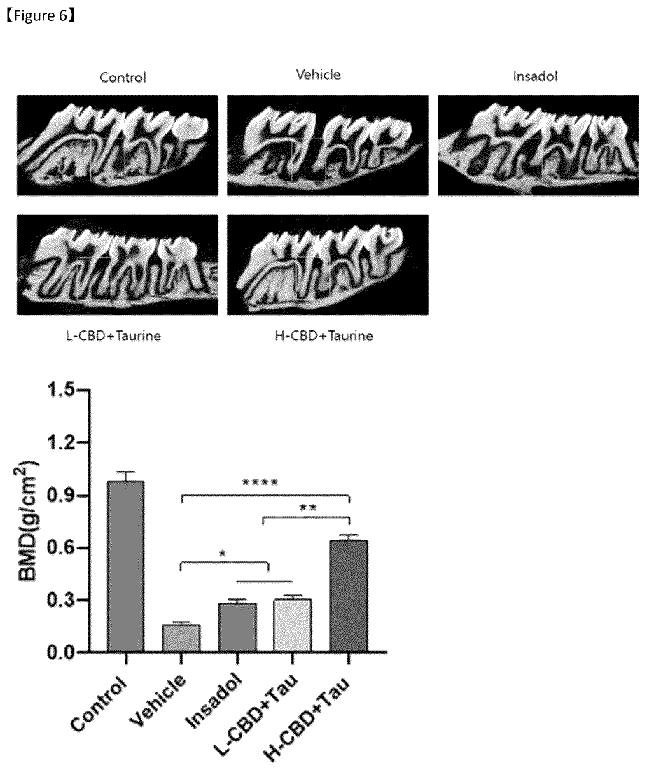

To examine the efficacy of co-administration of CBD and Taurine in inhibiting alveolar bone loss according to concentration, BMD (bone mineral density) was measured and the results are shown in FIG. 6. As a result of the measurement, the H-CBD+Taurine group showed the highest inhibition of BMD decrease, indicating that high concentrations of CBD and taurine have excellent periodontitis treatment efficacy when administered together (*, P<0.05; **, P<0.01; ***, P<0.001).

### MANUFACTURING EXAMPLES

Hereinafter, an example of manufacturing a pharmaceutical or food composition containing the above CBD and taurine as active ingredients according to the present invention was described. However, these examples are not intended to limit the present invention, but merely to illustrate it in detail. The pharmaceutical or food composition of Manufacturing Example 1 or 2 was prepared according to the following components and component ratio using the above active ingredients by conventional methods.

### [Manufacturing Example 1] Manufacturing of Pharmaceutical Composition

### <1-1> Manufacturing of Powder

CBD and Taurine 20 mg
Lactose 100 mg
Talc 10 mg

The above ingredients were mixed and filled into a sealed bag to prepare a powder.

### <1-2> Manufacturing of Tablet

CBD and Taurine 10 mg
Corn starch 100 mg
Lactose 100 mg
Magnesium stearate 2 mg

The above ingredients were mixed and tablets were prepared according to the conventional tablet preparation method.

### <1-3> Manufacturing of Capsules

CBD and Taurine 10 mg
Microcrystalline cellulose 3 mg
Lactose hydrate 14.8 mg
Magnesium stearate 0.2 mg

After mixing the ingredients, the mixture was filled into a gelatin capsule according to a conventional capsule manufacturing method to produce a capsule.

### <1-4> Manufacturing of Injections

CBD and Taurine 10 mg
Mannitol 180 mg
2974 mg of sterile distilled water for injection
Sodium hydrogen phosphate 26 mg

After mixing the ingredients, the injection was manufactured according to the usual method for manufacturing injections, with the above ingredient content per ampoule (2 mL).

### <1-5> Preparation of Liquid

CBD and Taurine 10 mg
10 mg of isomerized sugar
Mannitol 5 mg
Appropriate amount of purified water
Lemon flavor as needed

The liquid was prepared by adding the above ingredients to purified water in accordance with the usual manufacturing method, dissolving each ingredient, adding the appropriate amount of lemon flavor, adding purified water to adjust the total volume to 100 mL, and sterilizing it.

### [Manufacturing Example 2] Manufacturing of Health Functional Food

### <2-1> Manufacturing of Health Functional Food

CBD and Taurine 10 mg
Vitamin mixture Appropriate amount
Vitamin A acetate 70 μg
Vitamin E 1.0 mg
Vitamin B₁ 0.13 mg
Vitamin B₂ 0.15 mg
Vitamin B₆ 0.5 mg
Vitamin B₁₂ 0.2 μg
Vitamin C 10 mg
Biotin 10 μg
Nicotinamide 1.7 mg
Folic acid 50 μg
pantothenate 0.5 mg
Mineral mixture Appropriate amount
Ferrous sulfate 1.75 mg
Zinc oxide 0.82 mg
Magnesium carbonate 25.3 mg
Potassium phosphate monobasic 15 mg
2nd calcium phosphate 55 mg
Potassium citrate 30 mg
Calcium carbonate 100 mg
Magnesium chloride 24.8 mg

The component ratio of the above vitamin and mineral mixture is a desirable embodiment of a composition that is relatively suitable for health foods, but it is permissible to modify the composition ratio arbitrarily. The above ingredients can be mixed according to the usual method of manufacturing health functional food, and then granules can be manufactured and used to manufacture health functional food according to the normal method.

### <2-2> Manufacturing of Health Drinks

CBD and Taurine 10 mg
Vitamin C 15 g
Vitamin E (powder) 100 g
Ferrous lactate 19.75 g
Zinc oxide 3.5 g
Nicotinamide 3.5 g
Vitamin A 0.2 g
Vitamin B1 0.25 g
Vitamin B2 0.3 g
Purified water Appropriate amount

The above ingredients were mixed according to the normal health drink manufacturing method, and then the mixture was heated and stirred at 85° C. for about one hour. The resulting solution was filtered, collected in a sterilized 2-liter container, sealed and sterilized, and then refrigerated before being used to manufacture the health drink composition of the present invention.

The above composition ratio is a desirable embodiment of a composition that contains ingredients suitable for relatively flavored beverages, but it is permissible to arbitrarily modify the composition ratio according to regional and ethnic preferences, such as the demand group, country of demand, and use.

The description of the present invention given above is for illustrative purposes, and a person with ordinary knowledge in the technical field to which the present invention belongs will understand that the present invention can be easily transformed into other specific forms without changing its technical ideas or essential features. Therefore, the embodiments described above should be understood as illustrative in all respects and not as limiting.

1. A method for preventing or treating periodontitis, comprising administering or ingesting a composition comprising cannabidiol and taurine, or a pharmaceutically acceptable salt thereof, as active ingredients to a subject in need thereof.

2. The method for preventing or treating periodontitis of claim 1, wherein the cannabidiol is represented by the following chemical formula 1:

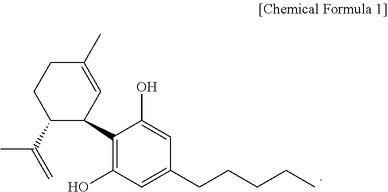 [SMILES: C=C(C)[C@@H]1CCC(C)=C[C@H]1c1c(O)cc(CCCCC)cc1O]

3. The method for preventing or treating periodontitis of claim 1, wherein the taurine is represented by the following chemical formula 2:

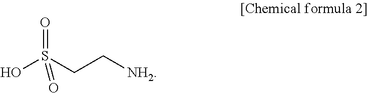

4. The method for preventing or treating periodontitis of claim 1, wherein the effective ingredient inhibits TNF-α and IL-1β.

5. A quasi-drug composition for preventing or ameliorating periodontitis containing cannabidiol and taurine, or pharmaceutically acceptable salts thereof, as active ingredients.

6. A food composition for preventing or ameliorating periodontitis, comprising cannabidiol and taurine, or a pharmaceutically acceptable salt thereof, as active ingredients.

7. (canceled)

8. (canceled)
